(19) United States
(12) Patent Application Publication
(10) Pub. No.: US 2026/0054069 A1
(43) Pub. Date: Feb. 26, 2026

(54) EVOKED BIOLOGICAL RESPONSE PROXIMITY ANALYSIS

(71) Applicant: Cochlear Limited, Macquarie University, NSW (AU)

(72) Inventors: Mattheus Johannes Petrus KILLIAN, Mechelen (BE); Christopher John JAMES, Toulouse (FR); Christopher Joseph LONG, Centennial, CO (US)

(21) Appl. No.: 19/104,847

(22) PCT Filed: Aug. 18, 2023
(86) PCT No.: PCT/IB2023/058295

§ 371 Date: Feb. 19, 2025

Related U.S. Application Data
(60) Provisional application No. 63/401,293, filed on Aug. 26, 2022.

(51) Int. Cl. A61N 1/36; A61N 1/372

(52) U.S. Cl. CPC A61N 1/36039; A61N 1/37217

(57) ABSTRACT

Presented herein are techniques for use of evoked biological responses (evoked responses), to determine relative proximity of electrodes to a target structure in a recipient. For example, in certain embodiments, the techniques presented herein use evoked biological responses to determine the relative proximity of electrodes to a modiolus of a cochlea.

### BACKGROUND

### Field of the Invention

The present invention relates generally to techniques for analysis of evoked biological responses to determine a relative proximity of electrodes to a target structure.

### Related Art

Medical devices have provided a wide range of therapeutic benefits to recipients over recent decades. Medical devices can include internal or implantable components/devices, external or wearable components/devices, or combinations thereof (e.g., a device having an external component communicating with an implantable component). Medical devices, such as traditional hearing aids, partially or fully-implantable hearing prostheses (e.g., bone conduction devices, mechanical stimulators, cochlear implants, etc.), pacemakers, defibrillators, functional electrical stimulation devices, and other medical devices, have been successful in performing lifesaving and/or lifestyle enhancement functions and/or recipient monitoring for a number of years.

The types of medical devices and the ranges of functions performed thereby have increased over the years. For example, many medical devices, sometimes referred to as “implantable medical devices,” now often include one or more instruments, apparatus, sensors, processors, controllers or other functional mechanical or electrical components that are permanently or temporarily implanted in a recipient. These functional devices are typically used to diagnose, prevent, monitor, treat, or manage a disease/injury or symptom thereof, or to investigate, replace or modify the anatomy or a physiological process. Many of these functional devices utilize power and/or data received from external devices that are part of, or operate in conjunction with, implantable components.

### SUMMARY

In one aspect, a method is provided. The method comprises: capturing a plurality of electrically evoked compound action potentials (ECAPs) via electrodes configured to be inserted into an inner ear; and estimating a proximity of at least one of the electrodes to a modiolus based on the plurality of ECAPs.

In another aspect, one or more non-transitory computer readable storage media comprising instructions are provided. The instructions, when executed by one or more processors, cause the one or more processors to: perform, with one or more of a plurality of electrodes configured to be inserted into an inner ear of a recipient, neural response telemetry measurements to obtain a plurality of evoked biological responses; and use the plurality of evoked biological responses to estimate a relative proximity of the electrodes to a wall of the inner ear.

In another aspect, a method is provided. The method comprises: obtaining, with one or more of a plurality of electrodes configured to be inserted into a recipient, a plurality of evoked biological responses; and analyzing the plurality of evoked biological responses relative to one another to determine a relative proximity of at least one of the electrodes to a target structure within the recipient.

In another aspect, a system is provided. The system comprises: one or more electrodes configured to record a plurality of evoked biological responses from a cochlea of a recipient; and one or more processors configured to analyze the plurality of evoked biological responses relative to one another to determine a relative modiolar proximity of the electrodes.

### BRIEF DESCRIPTION OF THE DRAWINGS

Embodiments of the present invention are described herein in conjunction with the accompanying drawings, in which:

FIG. 1A is a schematic diagram illustrating a cochlear implant system with which aspects of the techniques presented herein can be implemented;

FIG. 1B is a side view of a recipient wearing a sound processing unit of the cochlear implant system of FIG. 1A;

FIG. 1C is a schematic view of components of the cochlear implant system of FIG. 1A;

FIG. 1D is a block diagram of the cochlear implant system of FIG. 1A;

FIG. 1E is a schematic diagram illustrating a computing system with which aspects of the techniques presented herein can be implemented;

FIGS. 2A and 2B are schematic diagrams illustrating the impact of relative positioning of electrodes to a modiolus can have on spread of excitation;

FIG. 3 is a graph, shown in the time domain, illustrating a nominal/typical ECAP response obtained from an inner ear of a recipient in response to a biphasic stimulation signal;

FIG. 4 is a graph illustrating an example Spread Of Excitation (SOE) function determined from ECAP responses obtained from an inner ear of a recipient;

FIGS. 5A and 5B, are graphs illustrating example SOE functions where the peaks are shifted apically and/or basally from a probe electrode, in accordance with aspects presented herein;

FIGS. 6A, 6B, 6C, and 6D, are diagrams further illustrating the concept of a shifted or “off probe peak,” assuming a homogeneous spiral ganglion and scala, in accordance with aspects presented herein;

FIG. 7 is a graph illustrating that ECAP threshold profiles can vary over an electrode array, in accordance with aspects presented herein;

FIGS. 8A, 8B, and 8C schematically illustrate the effect of over-insertion and pull back of a stimulating assembly that can be initiated using the techniques presented herein;

FIG. 9 is a representation of a computed tomography (CT) image of an over-inserted so-called slim modiolar stimulating assembly, in accordance with aspects presented herein;

FIG. 10 illustrates an anticipated SOE curve, in accordance with aspects presented herein;

FIG. 11 is a graph illustrating ECAP amplitudes evoked by a probe pulse after a preceding masker pulse at the same electrode, in accordance with aspects presented herein;

FIGS. 12A, 12B, and 12C are images illustrating optimal positioning of a sheath during insertion of an electrode array into a plastic model of a cochlea;

FIG. 13 is an annotated image illustrating sub-optimal positioning of a sheath during insertion of an electrode array into a plastic model of a cochlea;

FIGS. 14A and 14B are schematic diagrams illustrating a sheath with electrodes disposed thereon, in accordance with certain embodiments presented herein;

FIG. 15 is a schematic diagrams illustrating a sheath with electrodes disposed thereon, where the sheath is in a sub-optimal position for insertion of an electrode array;

FIG. 16A is a schematic diagram illustrating a sheath with electrodes disposed thereon during a round window insertion, where the sheath is improperly angled for insertion of an electrode array;

FIG. 16B is a schematic diagram illustrating a sheath with electrodes disposed thereon during a round window insertion, where the sheath is correctly angled for insertion of an electrode array;

FIG. 16C is another schematic diagram illustrating a sheath with electrodes disposed thereon during a round window insertion, where the sheath is improperly angled for insertion of an electrode array;

FIG. 16D is a graph illustrating distance from modiolus versus angle of sheath for insertion shown in FIGS. 16A, 16B, and 16C;

FIG. 17A is a schematic diagram illustrating a sheath with electrodes disposed thereon during a cochleostomy insertion, where the sheath is improperly angled for insertion of an electrode array;

FIG. 17B is a schematic diagram illustrating a sheath with electrodes disposed thereon during a cochleostomy insertion, where the sheath is correctly angled for insertion of an electrode array;

FIG. 17C is another schematic diagram illustrating a sheath with electrodes disposed thereon during a cochleostomy insertion, where the sheath is improperly angled for insertion of an electrode array;

FIG. 17D is a graph illustrating distance from modiolus versus angle of sheath for insertion shown in FIGS. 17A, 17B, and 17C;

FIG. 18 is a functional block diagram illustrating an example evoked biological response analysis system configured to implement aspects of the techniques presented herein;

FIG. 19 is a schematic diagram illustrating a vestibular system with which aspects of the techniques presented herein can be implemented;

FIG. 20 is a flowchart of another example method, in accordance with certain embodiments presented herein;

FIG. 21 is a flowchart of another example method, in accordance with certain embodiments presented herein; and

FIG. 22 is a flowchart of another example method, in accordance with certain embodiments presented herein.

### DETAILED DESCRIPTION

Presented herein are techniques for use of evoked biological responses (evoked responses), to determine relative proximity of electrodes to a target structure in a recipient. For example, in certain embodiments, the techniques presented herein use evoked biological responses to determine the relative proximity of electrodes to a modiolus of a cochlea. The proximity of an electrode to a modiolus of a cochlea is referred to herein as the “modiolar proximity” of the electrode.

Merely for ease of description, the techniques presented herein are primarily described with reference to a specific medical device system, namely a cochlear implant system. However, it is to be appreciated that the techniques presented herein can also be partially or fully implemented by other types of medical device systems. For example, the techniques presented herein can be implemented by hearing aid systems and/or auditory prosthesis systems that include one or more other types of auditory prostheses, middle ear auditory prostheses, bone conduction devices, direct acoustic stimulators, electro-acoustic prostheses, auditory brain stimulators, combinations or variations thereof, etc. The techniques presented herein can also be implemented dedicated tinnitus therapy devices and tinnitus therapy device systems. In further embodiments, the presented herein can also be implemented by, or used in conjunction with, vestibular devices (e.g., vestibular implants), visual devices (i.e., bionic eyes), sensors, pacemakers, drug delivery systems, defibrillators, functional electrical stimulation devices, catheters, seizure devices (e.g., devices for monitoring and/or treating epileptic events), sleep apnea devices, electroporation devices, etc.

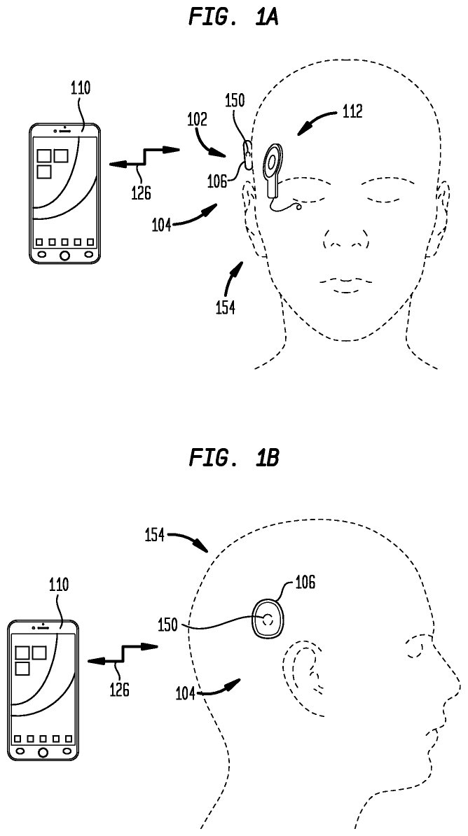

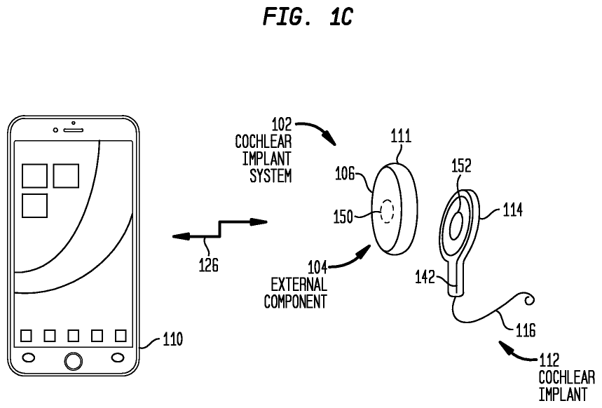

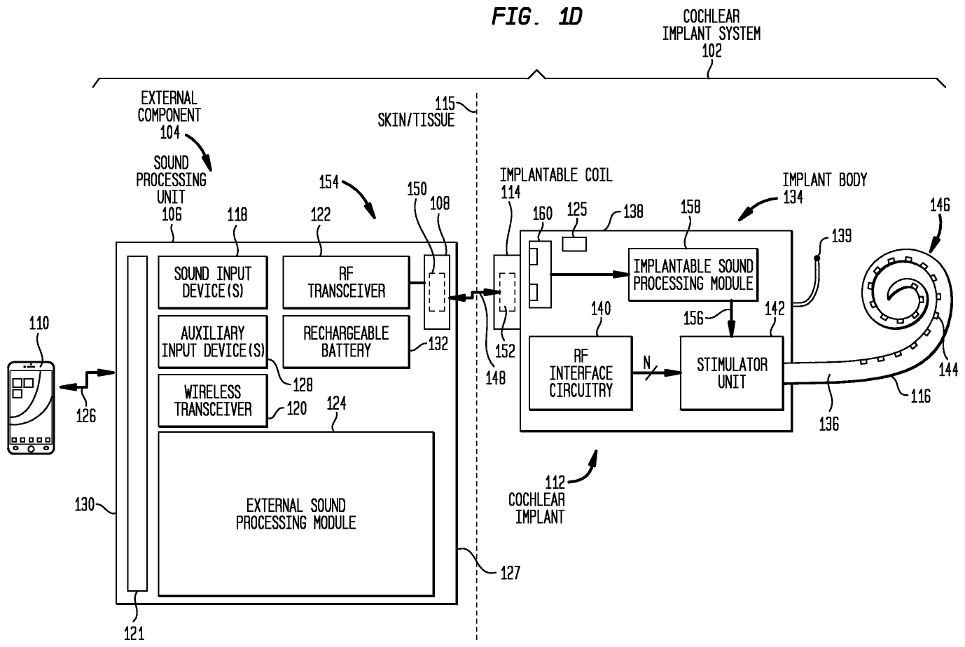

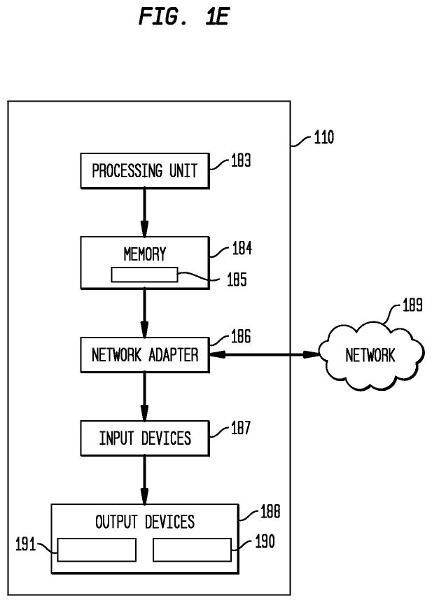

FIGS. 1A-IE illustrates an example cochlear implant system 102 with which aspects of the techniques presented herein can be implemented. The cochlear implant system 102 comprises an external component 104 and an implantable component 112. In the examples of FIGS. 1A-IE, the implantable component is sometimes referred to as a “cochlear implant.” FIG. 1A illustrates the cochlear implant 112 implanted in the head 154 of a user, while FIG. 1B is a schematic drawing of the external component 104 worn on the head 154 of the user. FIG. 1C is another schematic view of the cochlear implant system 102, while FIG. 1D illustrates further details of the cochlear implant system 102. For ease of description, FIGS. 1A-1E will generally be described together.

Cochlear implant system 102 includes an external component 104 that is configured to be directly or indirectly attached to the body of the user and an implantable component 112 configured to be implanted in the user. In the examples of FIGS. 1A-1E, the external component 104 comprises a sound processing unit 106, while the cochlear implant 112 includes an implantable coil 114, an implant body 134, and an elongate stimulating assembly 116 configured to be implanted in the user's cochlea.

In the example of FIGS. 1A-IE, the sound processing unit 106 is an off-the-ear (OTE) sound processing unit, sometimes referred to herein as an OTE component, which is configured to send data and power to the implantable component 112. In general, an OTE sound processing unit is a component having a generally cylindrically shaped housing 111 and which is configured to be magnetically coupled to the user's head (e.g., includes an integrated external magnet 150 configured to be magnetically coupled to an implantable magnet 152 in the implantable component 112). The OTE sound processing unit 106 also includes an integrated external (headpiece) coil 108 that is configured to be inductively coupled to the implantable coil 114.

It is to be appreciated that the OTE sound processing unit 106 is merely illustrative of the external devices that can operate with implantable component 112. For example, in alternative examples, the external component can comprise a behind-the-ear (BTE) sound processing unit or a micro-BTE sound processing unit and a separate external. In general, a BTE sound processing unit comprises a housing that is shaped to be worn on the outer ear of the user and is connected to the separate external coil assembly via a cable, where the external coil assembly is configured to be magnetically and inductively coupled to the implantable coil 114. It is also to be appreciated that alternative external components can be located in the user's ear canal, worn on the body, etc.

As noted above, the cochlear implant system 102 includes the sound processing unit 106 and the cochlear implant 112. However, as described further below, the cochlear implant 112 can operate independently from the sound processing unit 106, for at least a period, to stimulate the user. For example, the cochlear implant 112 can operate in a first general mode, sometimes referred to as an “external hearing mode,” in which the sound processing unit 106 captures sound signals which are then used as the basis for delivering stimulation signals to the user. The cochlear implant 112 can also operate in a second general mode, sometimes referred as an “invisible hearing” mode, in which the sound processing unit 106 is unable to provide sound signals to the cochlear implant 112 (e.g., the sound processing unit 106 is not present, the sound processing unit 106 is powered-off, the sound processing unit 106 is malfunctioning, etc.). As such, in the invisible hearing mode, the cochlear implant 112 captures sound signals itself via implantable sound sensors and then uses those sound signals as the basis for delivering stimulation signals to the user. Further details regarding operation of the cochlear implant 112 in the external hearing mode are provided below, followed by details regarding operation of the cochlear implant 112 in the invisible hearing mode. It is to be appreciated that reference to the external hearing mode and the invisible hearing mode is merely illustrative and that the cochlear implant 112 can also operate in alternative modes.

In FIGS. 1A and 1C, the cochlear implant system 102 is shown with an external computing device 110, configured to implement aspects of the techniques presented. The computing device 110, which is shown in greater detail in FIG. 1E, is, for example, a personal computer, server computer, hand-held device, laptop device, multiprocessor system, microprocessor-based system, programmable consumer electronic (e.g., smart phone), network PC, minicomputer, mainframe computer, tablet, remote control unit, distributed computing environment that include any of the above systems or devices, and the like. The computing device 110 can be a single virtual or physical device operating in a networked environment over communication links to one or more remote devices, such as an implantable medical device or implantable medical device system.

In its most basic configuration, computing device 110 includes at least one processing unit 183 and memory 184. The processing unit 183 includes one or more hardware or software processors (e.g., Central Processing Units) that can obtain and execute instructions. The processing unit 183 can communicate with and control the performance of other components of the computing system 110.

The memory 184 is one or more software or hardware-based computer-readable storage media operable to store information accessible by the processing unit 183. The memory 184 can store, among other things, instructions executable by the processing unit 183 to implement applications or cause performance of operations described herein, as well as other data. The memory 184 can be volatile memory (e.g., RAM), non-volatile memory (e.g., ROM), or combinations thereof. The memory 884 can include transitory memory or non-transitory memory. The memory 184 can also include one or more removable or non-removable storage devices. In examples, the memory 184 can include RAM, ROM, EEPROM (Electronically-Erasable Programmable Read-Only Memory), flash memory, optical disc storage, magnetic storage, solid state storage, or any other memory media usable to store information for later access. In examples, the memory 184 encompasses a modulated data signal (e.g., a signal that has one or more of its characteristics set or changed in such a manner as to encode information in the signal), such as a carrier wave or other transport mechanism and includes any information delivery media. By way of example, and not limitation, the memory 184 can include wired media such as a wired network or direct-wired connection, and wireless media such as acoustic, RF, infrared and other wireless media or combinations thereof. In certain embodiments, the memory 184 comprises environmental sound training logic 185 that, when executed, enables the processing unit 183 to perform aspects of the techniques presented.

In the illustrated example, the system 110 further includes a network adapter 186, one or more input devices 187, and one or more output devices 188. The system 110 can include other components, such as a system bus, component interfaces, a graphics system, a power source (e.g., a battery), among other components.

The network adapter 186 is a component of the computing system 110 that provides network access (e.g., access to at least one network 189). The network adapter 186 can provide wired or wireless network access and can support one or more of a variety of communication technologies and protocols, such as ETHERNET, cellular, BLUETOOTH, near-field communication, and RF (Radiofrequency), among others. The network adapter 186 can include one or more antennas and associated components configured for wireless communication according to one or more wireless communication technologies and protocols.

The one or more input devices 187 are devices over which the computing system 110 receives input from a user. The one or more input devices 187 can include physically-actuatable user-interface elements (e.g., buttons, switches, or dials), touch screens, keyboards, mice, pens, and voice input devices, among others input devices.

The one or more output devices 188 are devices by which the computing system 810 is able to provide output to a user. The output devices 188 can include, a display 190 and one or more speakers 191, among other output devices.

It is to be appreciated that the arrangement for computing system 110 shown in FIG. 1E is merely illustrative and that aspects of the techniques presented herein can be implemented at a number of different types of systems/devices. For example, the computing system 110 can be a laptop computer, tablet computer, mobile phone, surgical system, etc.

The OTE sound processing unit 106 comprises one or more input devices that are configured to receive input signals (e.g., sound or data signals). The one or more input devices include one or more sound input devices 118 (e.g., one or more external microphones, audio input ports, telecoils, etc.), one or more auxiliary input devices 128 (e.g., audio ports, such as a Direct Audio Input (DAI), data ports, such as a Universal Serial Bus (USB) port, cable port, etc.), and a wireless transmitter/receiver (transceiver) 120 (e.g., for communication with the external device 110). However, it is to be appreciated that one or more input devices can include additional types of input devices and/or less input devices (e.g., the wireless short range radio transceiver 120 and/or one or more auxiliary input devices 128 can be omitted).

The OTE sound processing unit 106 also comprises the external coil 108, a charging coil 121, a closely-coupled transmitter/receiver (RF transceiver) 122, sometimes referred to as or radio-frequency (RF) transceiver 122, at least one rechargeable battery 132, and an external sound processing module 124. The external sound processing module 124 can comprise, for example, one or more processors and a memory device (memory) that includes sound processing logic. The memory device can comprise any one or more of: Non-Volatile Memory (NVM), Ferroelectric Random Access Memory (FRAM), read only memory (ROM), random access memory (RAM), magnetic disk storage media devices, optical storage media devices, flash memory devices, electrical, optical, or other physical/tangible memory storage devices. The one or more processors are, for example, microprocessors or microcontrollers that execute instructions for the sound processing logic stored in memory device.

The implantable component 112 comprises an implant body (main module) 134, a lead region 136, and the intra-cochlear stimulating assembly 116, all configured to be implanted under the skin/tissue (tissue) 115 of the user. The implant body 134 generally comprises a hermetically-sealed housing 138 in which at least one battery 125, RF interface circuitry 140, and a stimulator unit 142 are disposed. The implant body 134 also includes the internal/implantable coil 114 that is generally external to the housing 138, but which is connected to the RF interface circuitry 140 via a hermetic feedthrough (not shown in FIG. 1D).

As noted, stimulating assembly 116 is configured to be at least partially implanted in the user's cochlea. Stimulating assembly 116 includes a plurality of longitudinally spaced intra-cochlear electrical stimulating contacts (electrodes) 144 that collectively form a contact or electrode array 146 for delivery of electrical stimulation (current) to the user's cochlea.

Stimulating assembly 116 extends through an opening in the user's cochlea (e.g., cochleostomy, the round window, etc.) and has a proximal end connected to stimulator unit 142 via lead region 136 and a hermetic feedthrough (not shown in FIG. 1D). Lead region 136 includes a plurality of conductors (wires) that electrically couple the electrodes 144 to the stimulator unit 142. The implantable component 112 also includes an electrode outside of the cochlea, sometimes referred to as the extra-cochlear electrode (ECE) 139.

As noted, the cochlear implant system 102 includes the external coil 108 and the implantable coil 114. The external magnet 152 is fixed relative to the external coil 108 and the implantable magnet 152 is fixed relative to the implantable coil 114. The magnets fixed relative to the external coil 108 and the implantable coil 114 facilitate the operational alignment of the external coil 108 with the implantable coil 114. This operational alignment of the coils enables the external component 104 to transmit data and power to the implantable component 112 via a closely-coupled wireless link 148 formed between the external coil 108 with the implantable coil 114. In certain examples, the closely-coupled wireless link 148 is a radio frequency (RF) link. However, various other types of energy transfer, such as infrared (IR), electromagnetic, capacitive and inductive transfer, can be used to transfer the power and/or data from an external component to an implantable component and, as such, FIG. 1D illustrates only one example arrangement.

As noted above, sound processing unit 106 includes the external sound processing module 124. The external sound processing module 124 is configured to convert received input signals (received at one or more of the input devices) into output signals for use in stimulating a first ear of a user (i.e., the external sound processing module 124 is configured to perform sound processing on input signals received at the sound processing unit 106). Stated differently, the one or more processors in the external sound processing module 124 are configured to execute sound processing logic in memory to convert the received input signals into output signals that represent electrical stimulation for delivery to the user.

As noted, FIG. 1D illustrates an embodiment in which the external sound processing module 124 in the sound processing unit 106 generates the output signals. In an alternative embodiment, the sound processing unit 106 can send less processed information (e.g., audio data) to the implantable component 112 and the sound processing operations (e.g., conversion of sounds to output signals) can be performed by a processor within the implantable component 112.

Returning to the specific example of FIG. 1D, the output signals are provided to the RF transceiver 122, which transcutaneously transfers the output signals (e.g., in an encoded manner) to the implantable component 112 via external coil 108 and implantable coil 114. That is, the output signals are received at the RF interface circuitry 140 via implantable coil 114 and provided to the stimulator unit 142. The stimulator unit 142 is configured to utilize the output signals to generate electrical stimulation signals (e.g., current signals) for delivery to the user's cochlea. In this way, cochlear implant system 102 electrically stimulates the user's auditory nerve cells, bypassing absent or defective hair cells that normally transduce acoustic vibrations into neural activity, in a manner that causes the user to perceive one or more components of the received sound signals.

As detailed above, in the external hearing mode the cochlear implant 112 receives processed sound signals from the sound processing unit 106. However, in the invisible hearing mode, the cochlear implant 112 is configured to capture and process sound signals for use in electrically stimulating the user's auditory nerve cells. In particular, as shown in FIG. 1D, the cochlear implant 112 includes a plurality of implantable sound sensors 160 and an implantable sound processing module 158. Similar to the external sound processing module 124, the implantable sound processing module 158 can comprise, for example, one or more processors and a memory device (memory) that includes sound processing logic. The memory device can comprise any one or more of. Non-Volatile Memory (NVM), Ferroelectric Random Access Memory (FRAM), read only memory (ROM), random access memory (RAM), magnetic disk storage media devices, optical storage media devices, flash memory devices, electrical, optical, or other physical/tangible memory storage devices. The one or more processors are, for example, microprocessors or microcontrollers that execute instructions for the sound processing logic stored in memory device.

In the invisible hearing mode, the implantable sound sensors 160 are configured to detect/capture signals (e.g., acoustic sound signals, vibrations, etc.), which are provided to the implantable sound processing module 158. The implantable sound processing module 158 is configured to convert received input signals (received at one or more of the implantable sound sensors 160) into output signals for use in stimulating the first ear of a user (i.e., the processing module 158 is configured to perform sound processing operations). Stated differently, the one or more processors in implantable sound processing module 158 are configured to execute sound processing logic in memory to convert the received input signals into output signals 156 that are provided to the stimulator unit 142. The stimulator unit 142 is configured to utilize the output signals 156 to generate electrical stimulation signals (e.g., current signals) for delivery to the user's cochlea, thereby bypassing the absent or defective hair cells that normally transduce acoustic vibrations into neural activity.

It is to be appreciated that the above description of the so-called external hearing mode and the so-called invisible hearing mode are merely illustrative and that the cochlear implant system 102 can operate differently in different embodiments. For example, in one alternative implementation of the external hearing mode, the cochlear implant 112 can use signals captured by the sound input devices 118 and the implantable sound sensors 160 in generating stimulation signals for delivery to the user.

Implantable medical devices, such as cochlear implant system 102, typically rely on various evoked biological responses (evoked responses) to determine settings/parameters for operation of the implantable medical device, where the term “evoked” is used herein in a manner synonymous with stimulation, namely an additional response (e.g., a response super-imposed on the basal response of the nerve) is created as a result of an input stimulation/stimulus. As such, there are existing techniques for determining/identifying the presence of an evoked biological response. For example, certain existing technologies exist for identification of Evoked Compound Action Potential (ECAP) responses, where the ECAP responses can be measured as the collective response of all neurons within a nerve or nerve portion to various stimulations. Neural response telemetry (NRT) is another term used in the art for measures of the responses of nerve cells to an evoked potential. Thus, recording an ECAP with a cochlear implant provides an objective measurement of the response of the auditory nerve to an electrical stimulus (as delivered by an implant electrode).

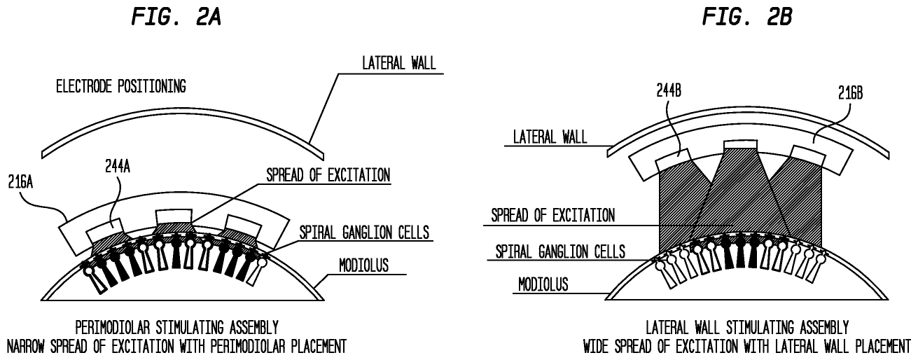

In general, the intracochlear position of the electrodes of a stimulating assembly, such as electrodes 144 of stimulating assembly 116, in relation to the modiolus is an important outcome of cochlear implant surgery. For example, as shown in FIGS. 2A and 2B, the positioning of the electrodes relative to the modiolus can have an impact on the amount of spread of excitation. More specifically, FIG. 2A illustrates a perimodiolar stimulating assembly 216A with electrodes 244A, while FIG. 2B illustrates a lateral wall stimulating assembly 216B with electrodes 244B. The electrodes 244A are shown positioned relatively closer to the modiolus (e.g., the target neurons) than the electrodes 244B. Due to this closer position, there is narrower spread of excitation from the electrical stimulation signals (current) delivered via electrodes 244A relative to the electrical stimulation signals (current) delivered via electrodes 244B when both electrodes stimulate at the same level within the dynamic range used for activation of the neural array, including the threshold level or comfort level. A narrower spread of excitation can be advantageous and enable improved hearing performance.

For ease of description, aspects of the techniques presented will be described with reference to analysis of a specific type of evoked biological response, namely an Evoked Compound Action Potential (ECAP) response. As described elsewhere herein, it is to be appreciated that the techniques can also be implemented to detect other types of evoked biological responses and can be implemented by a variety of implantable medical devices alone and/or in combination with one or more external devices.

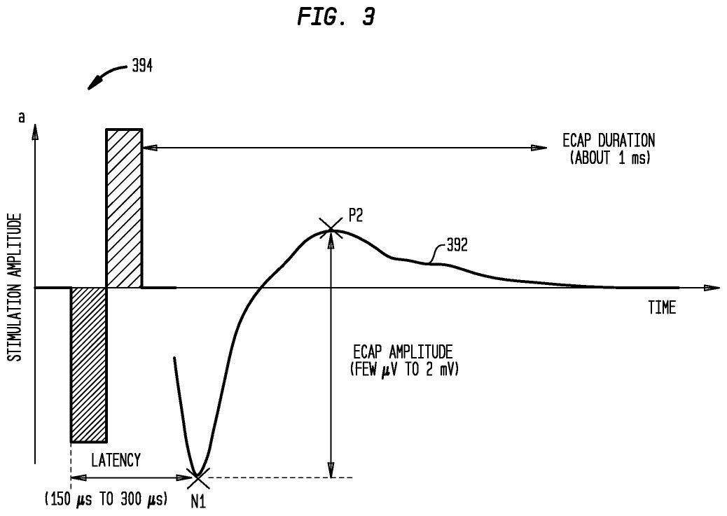

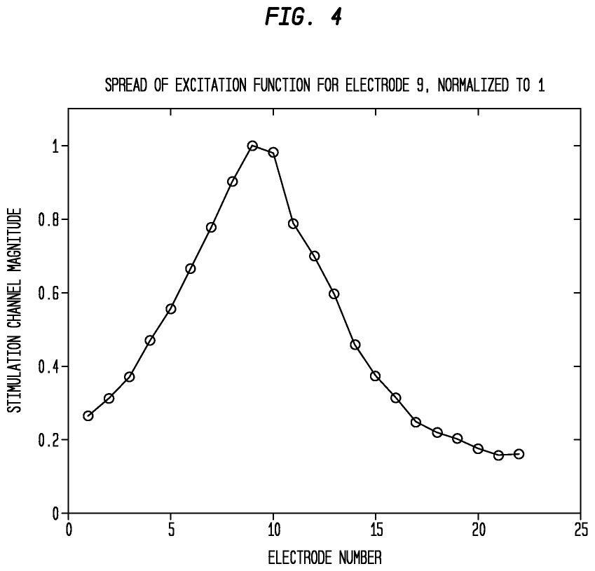

FIG. 3 is a graph, shown in the time domain, illustrating a nominal/typical ECAP response 392 captured from a cochlea of a recipient in response to a biphasic stimulation signal 394. That is, FIG. 3 schematically illustrates an ECAP measurement, including the stimulation amplitude, ECAP amplitude, and the ECAP duration. FIG. 4 is a graph illustrating an example Spread Of Excitation (SOE) function determined for electrode number nine (9) (probe electrode) of a twenty-two (22) electrode array, where the SOE function is normalized to one at the peak of the curve (1). Further details regarding Spread Of Excitation (SOE) functions and measurements can be found, for example, in “Spatial Spread of Neural Excitation in Cochlear Implant Recipients: Comparison of Improved ECAP Method and Psychophysical Forward Masking,” Cohen et al., Hearing Research 179 (2003) 72-87, the content of which is hereby incorporated by reference herein.

Presented herein are techniques for using evoked biological responses, potentially in combination with other information (e.g., electrical measurements, such as impedance, voltage, or current measurements), to determine a proximity of one or more electrodes to a target structure. In accordance with certain aspects, the techniques presented herein analyze the relative proximity of multiple (e.g., two or more) electrodes to a target structure, such as the modiolus of the cochlea. That is, certain embodiments determine/estimate the relative modiolar proximity of electrodes of an intracochlear stimulation assembly. The determined relative modiolar proximity can be used to optimize outcomes for the recipient (e.g., instruct surgeons or robot to act based on the determined modiolar proximity). In certain aspects, the techniques presented herein use ECAP threshold profiles and/or Spread Of Excitation (SOE) function to determine the relative modiolar proximity.

In certain embodiments presented herein, a system obtains multiple measurements, analyzes the plurality of measurements at different electrodes relative to one another to infer perimodiolar position (e.g., analyze the measurements relative to one another and infer proximity with the relative analysis). As described further below, the techniques presented herein can, for example, detect mid electrodes moving away from the modiolus due to over-insertion and inform the surgeon (or robotic system) to take a corrective action (e.g., pull back) to optimize perimodiolar position.

As noted above, certain aspects presented herein use Spread Of Excitation (SOE) functions to determine/estimate use in evaluating the relative proximity of a plurality of electrodes to a target structure within a recipient (e.g., evaluate the relative modiolar proximity of a plurality of electrodes). An SOE function is obtained by capturing and plotting ECAP response amplitudes at a plurality of different points, where the stimulation is delivered via a “probe” electrode and “masking” electrodes. For example, in a so-called forward-masking method, measurements are performed with the probe stimulation only (e.g., stimulation at the probe electrode only), measurements are performed with the masker stimulation only (e.g., without the probe stimulation), and then measurements are performed with both the masker stimulation and the probe stimulation. The measurements performed during these three measurement windows are then used to determine the “SOE function,” where entire process is referred to herein as the “SOE measurement (e.g., an SOE measurement is used to generate an SOE function).

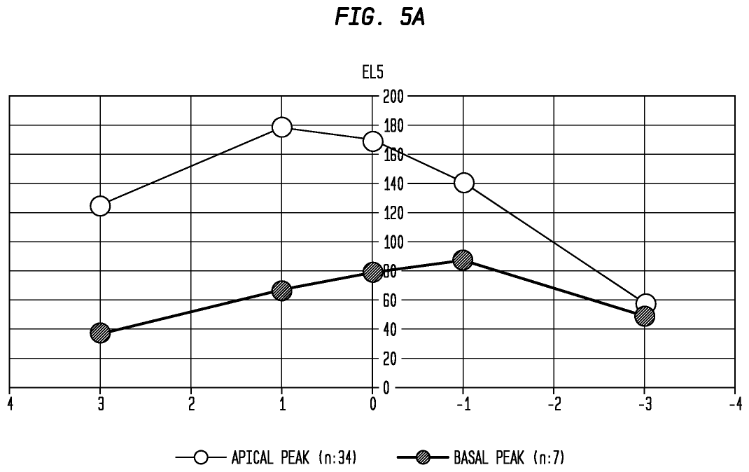

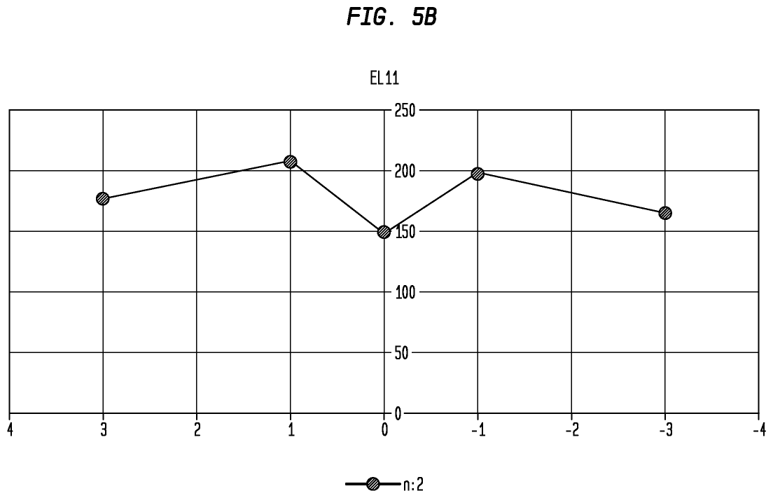

As shown in FIG. 4, an idealized SOE has the peak of excitation at the probe electrode. However, the present inventors have discovered that, in certain cases, the peaks can be shifted apically or basally with respect to probe electrode. In rare cases, there can be a double peak where peak are both basal and apical to the probe. FIGS. 5A and 5B are exemplary graphs illustrating example SOE functions where the peaks are shifted apically and/or basally.

More specifically, in FIGS. 5A and 5B, the probe electrode is in the middle (0) and the X-axis numbers show where the peak of excitation is, relative to the probe. The negative numbers indicate more basal positions, while the positive numbers indicate more apical positions. These graphs correspond to average SOE curves obtained with the probe current level equal to the masker current level. Table 1, below, is a summary of a test corresponding to FIGS. 5A and 5B, indicating the percentage at each an SOE peak was located at an electrode different from the probe electrode (e.g., how often a peak is detected apically versus basally with respect to probe). In Table 1, “n” is the number of SOE curves used for averaging.

TABLE 1

| EL | % peaks apically to Probe | % peaks basally to Probe |
|---|---|---|
| 17 (n: 191) | 37 | 5 |
| 11 (n: 203) | 48 | 5 |
| 5 (n: 192) | 27 | 6 |

As can be seen from Table 1, if there is a shift in the peak of excitation, the peak is most often shifted in the apical direction. The present inventors have recognized that this apical shift is most likely a result of a variability of distance to the spiral ganglion (modiolus). As such, the shift in peak can be used to estimate modiolar proximity, which is then can be used to setup a sequence of SOE peak measurements to provide an indication where the electrode is closest to the modiolus and where the electrode is further away from the modiolus.

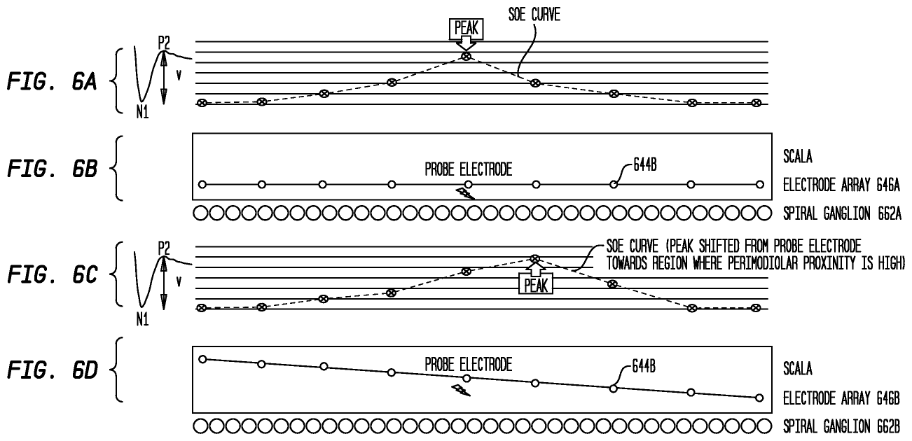

FIGS. 6A, 6B, 6C, and 6D, are diagrams further illustrating the concept of a shifted or “off probe peak,” assuming a homogeneous spiral ganglion and scala. In particular, FIG. 6B illustrates a portion of an electrode array 646A that comprises a plurality of electrodes 644A, where the electrodes 644 are all substantially the same distance from the spiral ganglion (modiolus) 662. FIG. 6A is a graph of an SOE function/curve generated from an SOE measurement performed via the electrode array 646A. Similarly, FIG. 6D illustrates a portion of an electrode array 646B that comprises a plurality of electrodes 644B, where the electrodes 644B are not same distance from the spiral ganglion (modiolus) 662. FIG. 6C is a graph of an SOE function/curve generated from an SOE measurement performed via the electrode array 646B.

A masker electrode that is closer to the modiolus (and thus spiral ganglion) than the probe electrode will be more effective in masking the activation field of the probe when stimulated at the same current level, leading to a larger ECAP amplitude. As such, while FIG. 6A illustrates a standard SOE function/curve where the peak of excitation is at the probe electrode, FIG. 6C illustrates an SOE function/curve where the peak is shifted apically, e.g., towards the electrodes located relatively closer to the modiolus. Therefore, in accordance with certain embodiments presented herein, the SOE functions obtained from a plurality of electrodes can be used to determine the relative modiolar proximity of one or more electrodes.

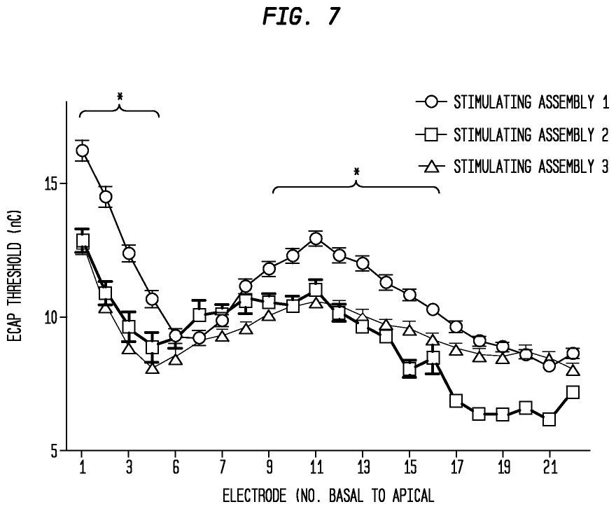

In certain embodiments presented herein, ECAP threshold profiles can be used to further optimize an estimate of modiolar proximity of one or more electrodes. For example, FIG. 7 is a graph illustrating ECAP threshold profiles for different stimulating assemblies having different properties such that the electrodes of the corresponding electrode arrays will each have at least a slightly different relative modiolar proximity (e.g., different final positions within the cochlea). FIG. 7 illustrates that the ECAP threshold profile can vary over the electrode array and that, in general, the lower the ECAP thresholds, the closer the electrode is to the modiolus. As such, the ECAP profile provides insight in relative closeness to the modiolus (e.g., ECAP threshold can be used to estimate relative modiolar proximity).

In FIG. 7, the increase in threshold at the base (lower electrode numbers) is noted and is likely due to electrode distance when the straight part of the stimulating assembly enters the cochlea with electrodes around electrode number 4-6 coming closer to the modiolus. Electrodes then tend to move away from the modiolus, particularly the electrodes of “straight” stimulating assemblies, as well as the electrodes of stimulating assemblies that over-inserted. At the apical end (highest electrode numbers) the electrodes come closer to the modiolus.

In certain examples, the ECAP threshold profile can be used as a first indication of modiolar proximity and/or can be used in addition to determine/define the most informative SOE measurement(s) at locations where a perimodiolar electrode is moving away from the modiolus. In certain examples, the ECAP threshold profile can be used as a weighting factor to correct the SOE curves, or can be used to set/adjust masker current levels to assure they all create a similar amount of neural activation.

In one specific implementation use case, the middle electrodes (e.g., the electrodes generally between the most apical electrodes and the most basal electrodes) move away from the modiolus when the electrode is over-inserted, which can be corrected by pulling back the electrode (e.g., by hand or with a robot when the surgeon makes use of a robot). As such, using the techniques described between, a surgical system can evaluate the relative modiolar proximity of the electrodes within the electrode array and determine when the stimulating assembly has been inserted (e.g., detect that the middle electrodes are relatively farther away from the modiolus than the apical and/or basal electrodes). As result, the surgical system can notify the surgeon, or instruct a robot, to make direct manipulations to the stimulating assembly.

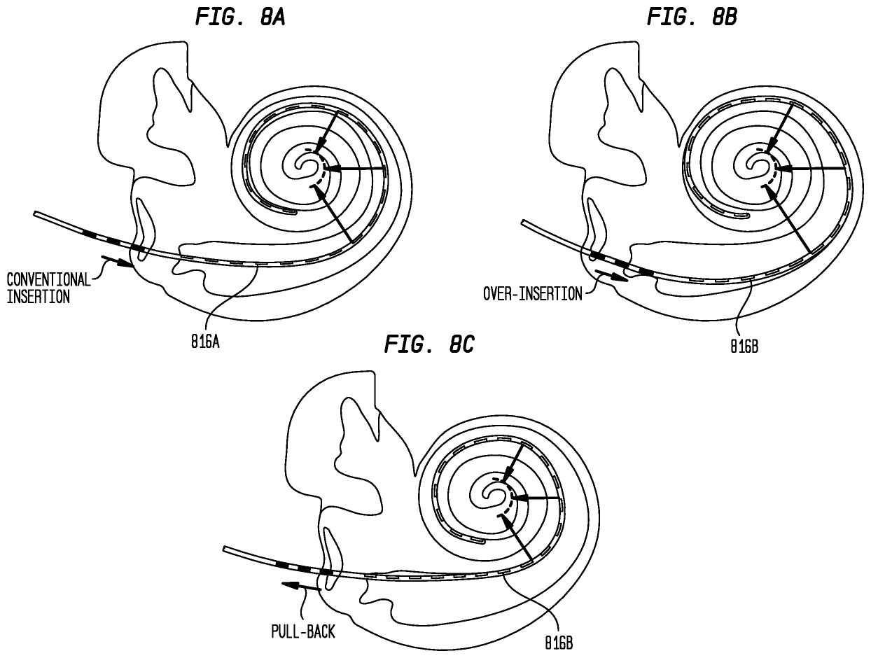

FIGS. 8A, 8B, and 8C schematically illustrate the effect of over-insertion and pull back of a stimulating assembly that can be initiated using the techniques presented herein. In FIGS. 8A, 8B, and 8C, the length of the arrows generally represent the distance between the electrodes and the modiolus.

FIG. 8A illustrates a conventional/standard insertion of a stimulating assembly 816A, while FIG. 8B illustrates an “over-insertion” of the stimulating assembly 816B. As noted above in the case of the over-inserted stimulating assembly 816B, the middle electrodes (e.g., the electrodes generally between the most apical electrodes and the most basal electrodes) move away from the modiolus. The positioning of the middle electrodes farther away from the modiolus, relative to the positioning of the apical and/or basal electrodes, can be determined using evoked biological responses captured and analyzed using the techniques presented herein. For example, the SOE spread could show the peak of the SOE function is moving towards the apical end (e.g., that the SOE peak is deviating from a known or desired location, determined based on prior data.), indicating that certain electrodes (e.g., the apical electrodes) are closer to the modiolus than the middle electrodes.

FIG. 8C illustrates the over-inserted stimulating assembly 816B of FIG. 8B following a so-called “pull-back” maneuver. As shown, following the pull-back maneuver, the middle electrodes are positioned closer to the modiolus than in the over-inserted position of FIG. 8B.

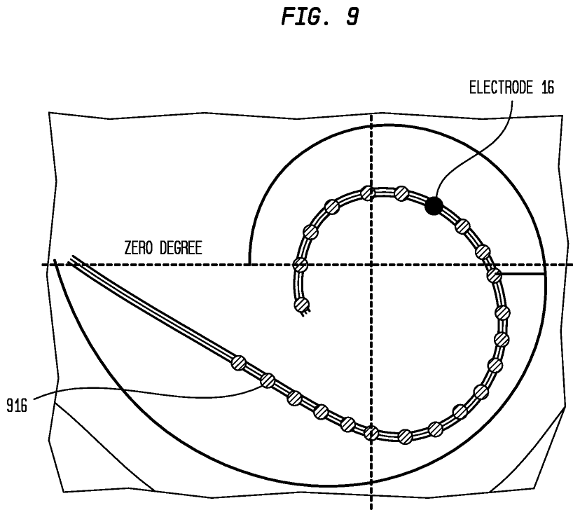

FIG. 9 is a representation of a computed tomography (CT) image of an over-inserted so-called slim modiolar stimulating assembly 916. As shown, the apical electrodes are generally in a modiolar position, but the mid electrodes are moving away from the modiolus. In this example, electrode 16 is where the electrode, moving from the apical to the basal direction, starts to move away from the modiolus. That is, deep insertion of certain stimulating assemblies push the electrodes in the middle of the electrode array away from the modiolus. The SOE measurements can be used to focus on this phenomenon (e.g., perform measurements where electrodes are anticipated to move away from the modiolus) and inform the surgeon on further manipulation of the electrode to obtain an optimal perimodiolar electrode position.

In one example relating to stimulating assembly 916 of FIG. 9, the over-insertion can be determined based on the ECAP threshold profile and one or more SOE measurements. For example, ECAPs can be recorded from the stimulating assembly 916 and the ECAP threshold profile can be generated and used provide a first indication of over insertion. For this, a set of data can be used in which the imaging data (e.g., CT imaging data) is correlated with a target ECAP threshold profile. Based on the correlation, an indicator can be set on the likelihood of over insertion. In certain embodiments, artificial intelligence (AI) can be used to identify the most prominent factors predicting over insertion and this can then be used in an algorithm.

When looking at ECAP threshold profiles for the slim modiolar stimulating assembly 916, it is possible to anticipate that the focus is on the mid electrodes moving away from the modiolus and there will be a peak in the threshold profile somewhere in the middle. How quickly the spread of excitation falls/rises around the peak (e.g., slopes of the ECAP threshold profile curve around the peak, or the extent of the spread of excitation) can also be an indicator. In addition, the position of the dip in the ECAP threshold profile (e.g., around electrode 4-6) can also be an indicator. Furthermore, it is anticipated that this dip can vary with insertion techniques making use of the round window or a cochleostomy and size/dimensions of the cochlea.

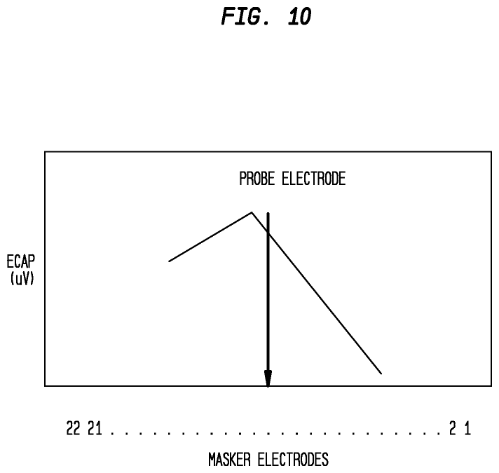

Based on the ECAP threshold profile, a SOE measurement can be set up where, with respect to the slim modiolar stimulating assembly 916 and starting at apex with electrode 22, electrode 16 is the position from where the electrodes starts to move away from modiolus in the basal direction. FIG. 10 illustrates the anticipated SOE curve with probe at electrode 16, where there is a shallow flank with potential offset peak at apical end and steep flank at basal end.

An offset peak and a given ratio in steepness of flanks can be an indication that the stimulating assembly 916 is over inserted. Electrical measurements, such as impedance measurements, current measurements, voltage measurements during stimulation (e.g., EVT measurements, transimpedance matrix (TIM) measurements, etc.) can be used to check to what extent the voltage spread is asymmetrical as this could be the reason for the non-symmetrical SOE curve. In certain embodiments, the voltage spread can be used to normalize (weight) the SOE curve to improve the sensitivity to detect to what extend the shape of the SOE curve is a predictor of modiolar distance.

In addition, the techniques presented herein can factor in the recording electrode which could be a reason for the observed SOE curve and/or could make use of the Amplitude Growth Functions (AGF) of the ECAP with respect to the current level of the stimulus. For example, the ECAP amplitude growth functions can be used to check for neural health, which in turn can be used to normalize the ECAP threshold and SOE curves to deduce from them modiolar distance (e.g., ECAP AGF functions are steeper when there is good neural survival).

In certain embodiments, the techniques presented herein control for neural factors, potentially, via any of a set of measurements of neural health (e.g., Electrocochleography (ECochG), ECAP thresholds, IPG effect, polarity effect, focused thresholds minus monopolar thresholds). The control can be accomplished, for example, by normalizing the ECAP measurements (Threshold profile, SOE, AGF, etc.) against the neural health measurements.

In addition, it may be the case that AGFs obtained with an electrode close to the modiolus are steeper than those obtained with an electrode further away from the modiolus. This can be used to detect whether manipulations of the electrode lead to closer modiolar proximity.

As noted, the techniques presented herein can use evoked biological responses (ECAPs) and data derived therefrom (e.g., SOE functions, ECAP threshold profiles, etc.) in combination with additional information. In certain embodiments, the additional information can include electrical measurements obtained via the electrodes positioned within the recipient, such as impedance measurements, current measurements, voltage measurements, etc. For example, as noted above, it is assumed that SOE peak shifts are a result of relative modiolar proximity. However, there could be other reasons for the shift (e.g., neural survival). In certain aspects, the additional data (e.g., EVT measurements and/or some neural survival measurement) could be used to confirm that the shift is actually due to modiolar proximity and not some other root cause (e.g., other measurements are used to eliminate other possible causes).

In further aspects, the additional information used with the evoked biological responses (ECAPs) and/or data derived therefrom (e.g., SOE functions, ECAP threshold profiles, etc.) could include normative population data (e.g., analyze a plurality of ECAPs relative to normative population data estimate the modiolar proximity). The normative population data include, for example, imaging data (possibly combined with modeling) for evaluation of perimodiolar position. In addition, there can be several normative sets, e.g., one for a perimodiolar electrode with optimal close perimodiolar position and one for a lateral wall electrode that is away from the modiolus. In certain such examples, the evoked biological responses are analyzed relative to the normative data (e.g., data indicating close to modiolus) and greater more deviation from the “good” average (prior population data set) can be used to infer modiolar proximity.

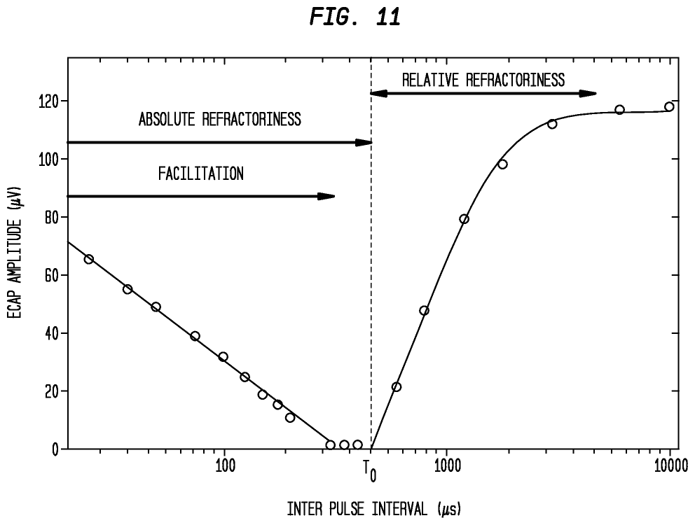

In certain embodiments, the ECAP responses to a stimulation pulse can be facilitated by a preceding pulse. For example, FIG. 11 shows ECAP amplitudes evoked by a probe pulse after a preceding masker pulse at the same electrode. The facilitation effect occurs at short inter pulse intervals.

The present inventors have projected that this facilitation effect is larger when an electrode is closer to the modiolus, thus this facilitated can be used to, for example, detect whether manipulations of the stimulation assembly lead to improved modiolar proximity. The preceding masker pulse can also be positioned at an electrode close to the probe electrode and the effect is expected to be larger when the masker electrode is closer to the modiolus. This effect can be used to detect whether a manipulation of the electrode leads to closer proximity of the Probe electrode.

All above or other measures, individually or in concert (e.g., based on the outcome of an Artificial Intelligence based algorithm), can be used to instruct the surgeon to manipulate the stimulating assembly to obtain the best possible modiolar proximity, e.g. pulling back an over-inserted stimulating assembly to get the mid electrode close to the modiolus, inserting the stimulating assembly deeper to assure the basal electrodes get closer to the modiolus, twisting the stimulating assembly to assure the precurved stimulating assembly puts itself in the ‘most convenient/least mechanically stressed’ position, etc.

A stimulating assembly can be inserted manually by surgeon or via a partially or fully automated process using a robotic system (e.g., partially or fully automated robotic system). In accordance with certain embodiments, the robotic system can manipulate the stimulating assembly (as indicated above) based on the above measures to achieve a best possible perimodiolar position of the stimulating assembly.

The above embodiments have generally been described with reference to evoked potentials, such as electrically evoked compound action potentials (ECAPs), captured via electrodes of a stimulating assembly at least partially inserted into the cochlea of a recipient. However, in certain embodiments, the evoked potentials can also or alternatively be captured via electrodes positioned an auxiliary/supplemental component used during the insertion process. Such auxiliary components can include, for example, an insertion tool, an insertion sheath, a probe assembly, etc.

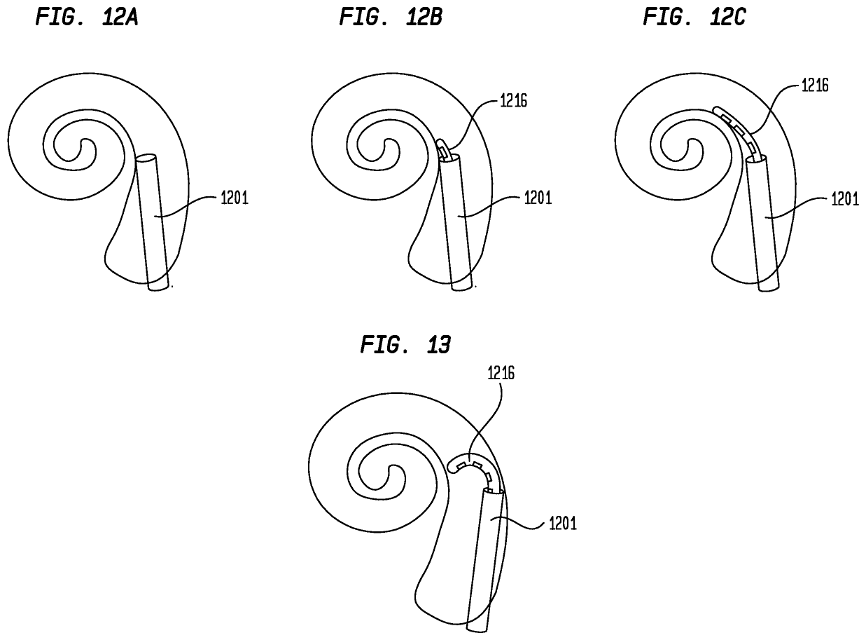

For example, pre-curved stimulating assemblies are generally inserted using a stiffening sheath that maintains the electrode array in a straight arrangement prior to insertion, then is removed during insertion. The position of the sheath in the cochlea can be important to ensuring a smooth insertion. Ideally, the tip of the sheath is positioned close to the modiolus so that the electrode array runs smoothly onto the modiolar wall as it exits the sheath. FIGS. 12A-12C illustrative correct positioning of a sheath 1201 during insertion of an electrode array 1216. However, as shown in FIG. 13, if the sheath 1201 is held with the tip toward the lateral wall, then in the case of a wide lumen, the stimulating assembly 1216 can bend over immediately on exiting the sheath 1201 and catch on the modiolus close to the sheath tip.

A solution to this issue is to include electrodes on the sheath and apply the same measurement technique as described above to determine the distance of the sheath from the modiolus and/or lateral wall. That is, by incorporating electrodes on the sheath it is possible to estimate the position of the sheath in the cochlea relative to the modiolus. If the tip is not sufficiently close to the modiolus, this could indicate that the sheath is in a sub-optimal angle or depth and hence pose a risk of complications during insertion. A system could monitor the position of the sheath relative to the modiolus and lateral wall (and thus the position in the cochlear duct) and indicate to the surgeon whether the sheath is being held at an optimal angle and depth. Note that the sheath is intended to be held in a static position during deployment of the electrode array, however it is known that sometimes surgeons unintentionally change the angle or depth of the sheath during deployment of the electrode. A system could continuously monitor the position of the sheath and alert the surgeon of an unintended change. These feedback signals could be provided to a surgeon or a robot performing robotic insertion of electrode array. In the case of a robot, a closed loop feedback system could be implemented to manage the optimal positioning of the sheath and insertion of the electrode.

In certain examples, the measurements are made using an electrode located at the tip of the sheath and/or the most apical electrode of the electrode array. When such electrode(s) contact the modiolus, it is anticipated that a large ECAP response will be detected, and that the ECAP response will decrease in amplitude as the electrode(s) move away. This could be the single signal to focus on and provide direct feedback to the surgeon during insertion and manipulation of the sheath (e.g., insertion angle, insertion depth).

In certain embodiments, transimpedance matrix measurements can be also or alternatively be used. In addition, multipolar stimulation be used to provide “virtual” sheath contacts.

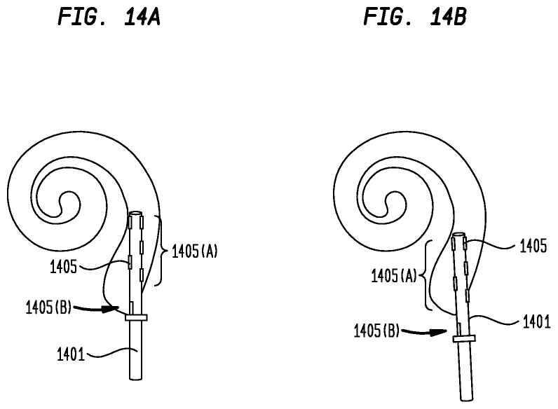

FIGS. 14A and 14B illustrate a sheath 1401 with electrodes 1405 disposed therein. FIG. 14A illustrates the sheath 1401 in an optimal/preferred position, while FIG. 14B illustrates the sheath 1401 not fully inserted. The arrangement of FIG. 14B could be detected and signaled to the surgeon (or a robot) to adjust the angle and depth of the sheath to optimize the insertion before or while advancing the electrode array out of the sheath 1401. In one example, the electrodes 1405(A) can be used to capture evoked biological responses that, as described above, can be used to determine if the tip of the sheath 1401 is in the optimal position close to the modiolus. In certain such examples, the impedance of electrode 1405(B) can be used to confirm that the sheath 1401 is fully inserted with the stopper resting on the cochlea. If the sheath 1401 is accidentally held outside the cochlea as shown in FIG. 14B, then electrode 1405(B) would lose connection with the cochlea fluid and go to high impedance.

It is noted that it is possible to measure sheath location with electrodes on one side of the sheath. However, it is also advantageous to include electrodes on either side (medial and lateral sides) of the sheath as shown in FIGS. 14A and 14B to improve the accuracy of estimate of the relative position within the cochlea duct since the algorithm detects the volume of the space immediately adjacent to the electrode and thus can determine the relative distance to the nearest wall with greater accuracy.

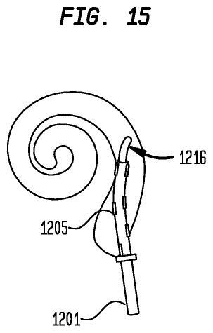

In certain examples, a sheath is held too hard against the modiolus, which can bend or kink the sheath. This can jam the stimulating assembly in the sheath preventing insertion, as shown in FIG. 15. A bend in the sheath can also cause the electrode array to twist and exit the sheath in a trajectory toward the lateral wall and potentially impacting on the lateral wall. Using the techniques presented herein, the measurement of the distance from the modiolus and lateral wall can be used to detect that the electrode array is not in the expected position as it deploys from the sheath.

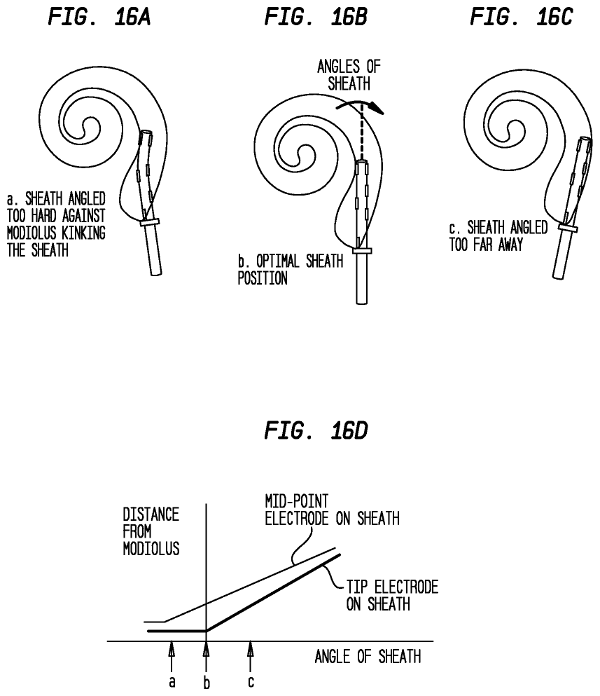

To determine if the sheath is in an optimal position, an algorithm may be applied based on the model depicted in FIGS. 16A-16D. FIGS. 16A, 16B, and 16C illustrate three positions of sheath when inserted into the cochlea via the round window. In operation, the surgeon can move the angle of the sheath from low to high and back again to traverse the space. The measurement system can then record the spacing of the tip and mid-point electrode to the modiolus and estimate the optimal angle where the tip electrode has just reached close proximity to the modiolus, and the mid-point electrode is spaced by some reasonable distance. An algorithm based on the curves shown in FIG. 16D can be used to determine whether the angle of the sheath has been optimized.

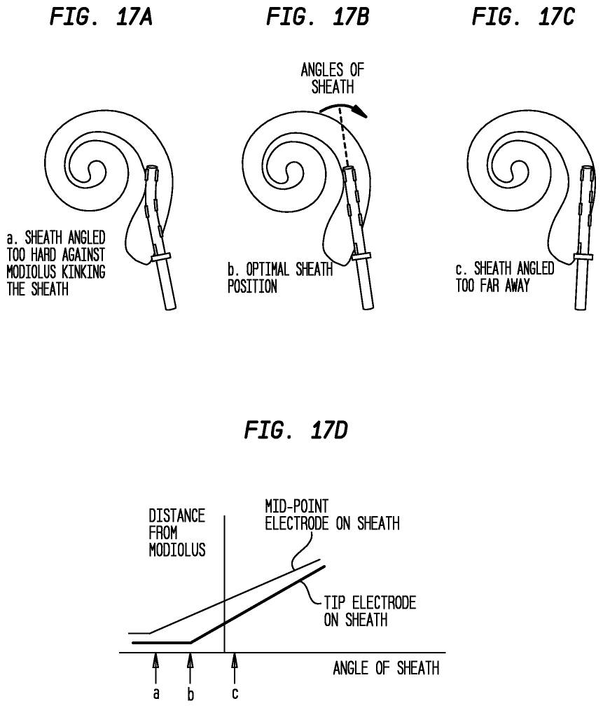

The model of the curves shown in FIGS. 16A-16D can also be applied for electrodes inserted through a cochleostomy. If inserted through a cochleostomy, the angles for the three cases would be modified by an amount depending on the exact position of the cochleostomy as shown in FIGS. 17A-17D. In general, the absolute angle of each of the three positions shown in FIGS. 17A, 17B, and 17C would be further anticlockwise relative to the orientation of the cochlea, however the difference in angle between FIGS. 17A, 17B, and 17C would be similar for cochleostomy to that of round window insertion. Again, an algorithm based on the curves shown in FIG. 17D can be used to determine whether the angle of the sheath has been optimized.

As noted, FIGS. 12A-17D illustrate concepts using electrodes attached to the sheath. It is to be appreciated that number of implementations are possible with this arrangement. For example, in certain embodiments, electrodes on the sheath may be connected back to a measurement system by wires or the electrodes could be connected to a small wireless transmitter incorporated in the sheath to transmit the information back without the inconvenience of a wired connection. In certain embodiments, the electrodes on the sheath could be connected to the electrodes of the intracochlear electrode array by means of brushes on the inside of the sheath to connect to the contacts of the electrode array. Moreover, instead of incorporating electrodes on the sheath, the contacts of the electrode array could be utilized simply by creating an opening in the sheath over a number of the contacts. Yet a further refinement of the above is to add a strain gauge to the sheath to detect whether it has been deformed. The signal from the strain gauge can be included with analysis of the position of the electrode array to further improve the potential to detect sub-optimal sheath position.

It is to be appreciated that straight (e.g., not pre-curved) electrode array insertions can be monitored in a similar manner to that described for perimodiolar stimulating insertions. The characteristic trajectory of a straight electrode array is known. By applying a detailed knowledge of the optimal insertion trajectory with known sub-optimal trajectories, it is possible to determine when the stimulating is deviating from the optimal insertion trajectory.

It is to be appreciated that the use of sheaths and measurements via electrodes of a stimulating assembly are not mutually exclusive and that the various embodiments described above can be combined in different arrangements. For example, a sheath without electrodes thereon can be used with a stimulating assembly that is used to obtain evoked biological responses (e.g., the sheath should include a conductive part or hole at the tip to assure evoked biological response measurements are possible). Other embodiments can make use of electrodes on both the sheath and the stimulating assembly to obtain evoked biological responses.

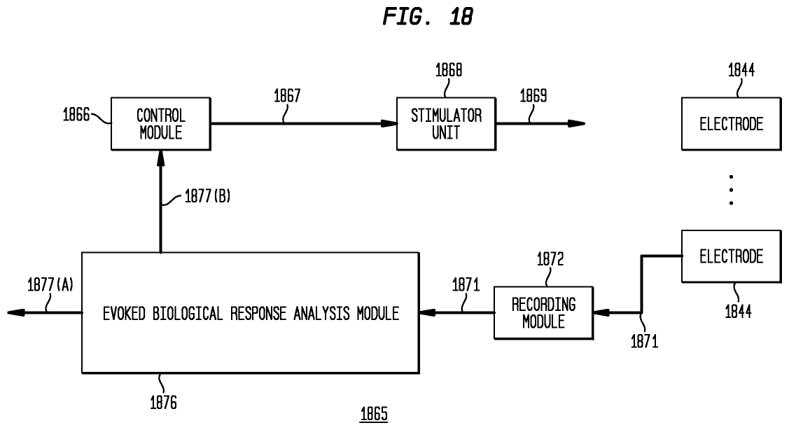

As noted above, aspects of the techniques presented herein capture/record evoked biological responses (e.g., ECAPs) from a recipient for use in evaluating the relative proximity of a plurality of electrodes to a target structure within a recipient (e.g., evaluate the relative modiolar proximity of a plurality of electrodes). FIG. 18 is a functional block diagram illustrating an example evoked biological response analysis system 1865 configured to implement aspects of the techniques presented herein. The evoked biological response analysis system 1865 can be implemented, for example, by an implantable medical device (e.g., cochlear implant 102), by the combination of an implantable medical device and an external device (e.g., cochlear implant 102 and external device 110), multiple implantable medical devices, multiple implantable medical devices and multiple external devices, etc.

As shown, the evoked biological response analysis system 1865 comprises a control module 1866, a stimulator unit 1868, a plurality of electrodes 1844, a recording module 1872, and an evoked biological response analysis module 1876. Again, for ease of illustration, FIG. 18 will be described with reference to analysis of ECAP responses. As described elsewhere herein, it is to be appreciated that the evoked biological response analysis system 1865, or a similar system, can be used to detect other types of evoked biological responses and can be implemented by a variety of implantable medical devices alone and/or in combination with one or more external devices. In addition, the evoked biological response analysis system 1865 can also be used to capture/record, for example, impedance or voltage measurements for use in evaluating, for example, the modiolar proximity of electrodes.

In the specific illustrative ECAP example of FIG. 18, the system 1865 is configured to capture/record a plurality of sets of electrophysiological signals from tissue of the recipient. In certain examples, a medical professional (e.g., clinician, audiologist, etc.) selects one or more of the plurality electrodes 1844, which are disposed within the cochlea of the recipient, for evaluation. The control module 1866 provides a control signal 1867 to the stimulator unit 1868 that causes the stimulator unit 1868 to deliver electrical stimulation signals 1869 to the recipient via, for example, the selected one or more electrodes 1844, or another electrode in close proximity to the selected one or more electrodes 1844, to perform one or more ECAP measurements.

Following delivery of the electrical stimulation signals 1869, an electrophysiological trace 1871 (i.e., a set of electrophysiological signals representing the electrical activity from the stimulated tissue) is recorded from the tissues by the recording module 1872. The recording module 1872 can be configured to, among other operations, amplify the electrophysiological trace 1871. The electrophysiological trace 1871 is, when captured via the recording module 1872 is provided to the evoked biological response analysis module 1876.

As noted above, the stimulation signals for performing an ECAP measurement can be complex. As such, the above process of delivering stimulation signals to the recipient and recording of a resulting electrophysiological trace 1871 can be repeated a number of times (e.g., with different stimulating and/or recording electrodes) and each electrophysiological trace 1871 can relate to a separate window of time, referred to as an epoch. It is to be appreciated that the stimulation signals can use a variety of different pulse shapes, such as rectangular pulses, ramped pulses, rhomboid pulses, curved pulses (e.g., sine waves), etc.

The evoked biological response analysis module 1876 is configured use a plurality of electrophysiological traces 1871 forming the ECAP responses to, as described elsewhere herein, evaluate the evoked biological response of one or more of the electrodes 1844, as described elsewhere herein. The evoked biological response analysis module 1876 can generate one or more outputs 1877 representative of the final classification/determination of the electrode proximity (e.g., modiolar proximity). In certain embodiments, the one or more outputs can be audible or visible outputs, represented by arrow 1877(A). In other embodiments, as described further below, the one or more outputs can be control outputs, represented by arrow 1877(B), that are sent to, for example, the control module 1866 for use as part of a closed-loop control system.

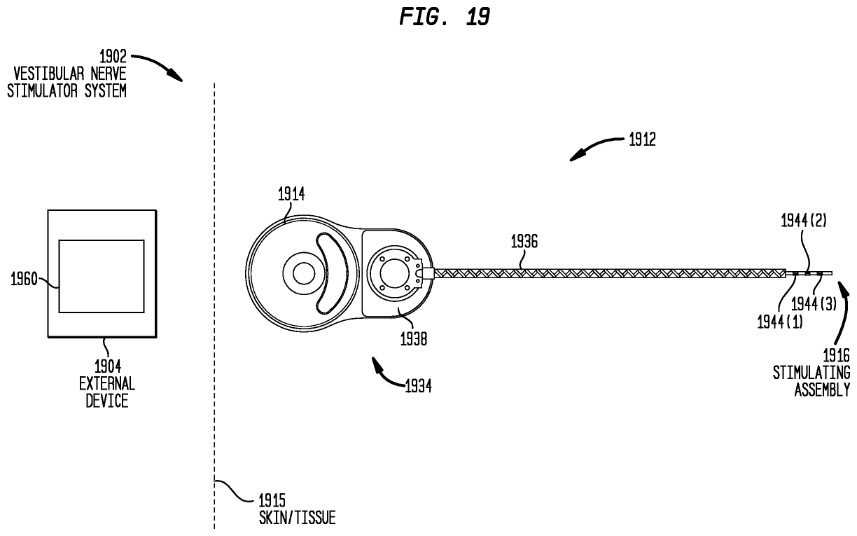

As previously described, the technology disclosed herein can be applied in any of a variety of circumstances and with a variety of different devices. One example device that can benefit from technology disclosed herein is described in more detail in FIG. 19. In particular, FIG. 19 illustrates an example vestibular stimulator system 1902, with which embodiments presented herein can be implemented. As shown, the vestibular stimulator system 1902 comprises an implantable component (vestibular stimulator) 1912 and an external device/component 1904 (e.g., external processing device, battery charger, remote control, etc.). The external device 1904 comprises a transceiver unit 1960. As such, the external device 1904 is configured to transfer data (and potentially power) to the vestibular stimulator 1912.

The vestibular stimulator 1912 comprises an implant body (main module) 1934, a lead region 1936, and a stimulating assembly 1916, all configured to be implanted under the skin/tissue (tissue) 1915 of the recipient. The implant body 1934 generally comprises a hermetically-sealed housing 1938 in which RF interface circuitry, one or more rechargeable batteries, one or more processors, and a stimulator unit are disposed. The implant body 134 also includes an internal/implantable coil 1914 that is generally external to the housing 1938, but which is connected to the transceiver via a hermetic feedthrough (not shown).

The stimulating assembly 1916 comprises a plurality of electrodes 1944 disposed in a carrier member (e.g., a flexible silicone body). In this specific example, the stimulating assembly 1916 comprises three (3) stimulation electrodes, referred to as stimulation electrodes 1944(1), 1944(2), and 1944(3). The stimulation electrodes 1944(1), 1944(2), and 1944(3) function as an electrical interface for delivery of electrical stimulation signals to the recipient's vestibular system.

The stimulating assembly 1916 is configured such that a surgeon can implant the stimulating assembly adjacent the recipient's otolith organs via, for example, the recipient's oval window. It is to be appreciated that this specific embodiment with three stimulation electrodes is merely illustrative and that the techniques presented herein may be used with stimulating assemblies having different numbers of stimulation electrodes, stimulating assemblies having different lengths, etc.

In operation, the vestibular stimulator 1912, the external device 1904, and/or another external device, can be configured to implement the techniques presented herein. That is, the vestibular stimulator 1912, possibly in combination with the external device 1904 and/or another external device, can include an evoked biological response analysis system, as described elsewhere herein.

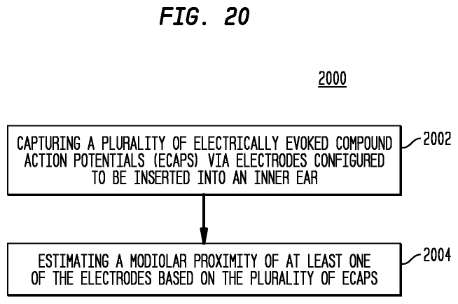

FIG. 20 is a flowchart of an example method 2000, in accordance with certain embodiments presented herein. Method 2000 begins at 2002 where a plurality of electrically evoked compound action potentials (ECAPs) are captured via electrodes configured to be inserted into an inner ear. At 2004, a system estimates a modiolar proximity of at least one of the electrodes based on the plurality of ECAPs.

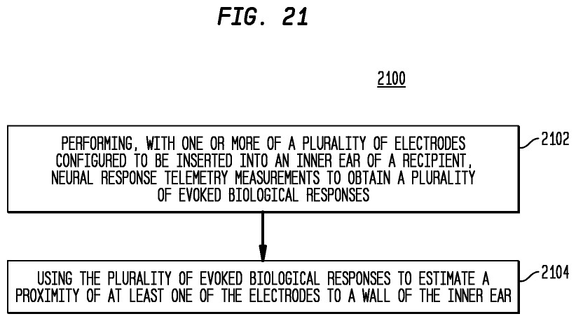

FIG. 21 is a flowchart of another example method 2100, in accordance with certain embodiments presented herein. Method 2100 begins at 2102 where a system performs, with one or more of a plurality of electrodes configured to be inserted into an inner ear of a recipient, neural response telemetry measurements to obtain a plurality of evoked biological responses. At 2104, the plurality of evoked biological responses are used to estimate a proximity of at least one of the electrodes to a wall of the inner ear.

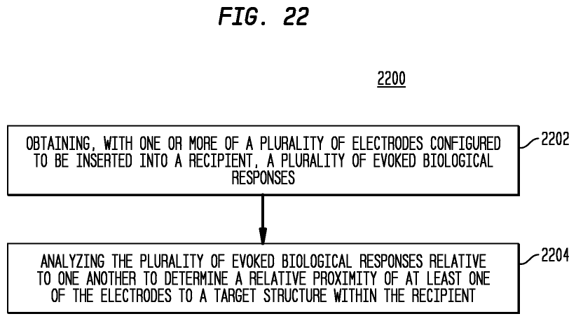

FIG. 22 is a flowchart of another example method 2200, in accordance with certain embodiments presented herein. Method 2200 begins at 2202 where a system obtains, with one or more of a plurality of electrodes configured to be inserted into a recipient, plurality of evoked biological responses. At 2204, the system analyzes the plurality of evoked biological responses relative to one another to determine a relative proximity of at least one of the electrodes to a target structure within the recipient.

As should be appreciated, while particular uses of the technology have been illustrated and discussed above, the disclosed technology can be used with a variety of devices in accordance with many examples of the technology. The above discussion is not meant to suggest that the disclosed technology is only suitable for implementation within systems akin to that illustrated in the figures. In general, additional configurations can be used to practice the processes and systems herein and/or some aspects described can be excluded without departing from the processes and systems disclosed herein.

This disclosure described some aspects of the present technology with reference to the accompanying drawings, in which only some of the possible aspects were shown. Other aspects can, however, be embodied in many different forms and should not be construed as limited to the aspects set forth herein. Rather, these aspects were provided so that this disclosure was thorough and complete and fully conveyed the scope of the possible aspects to those skilled in the art.

As should be appreciated, the various aspects (e.g., portions, components, etc.) described with respect to the figures herein are not intended to limit the systems and processes to the particular aspects described. Accordingly, additional configurations can be used to practice the methods and systems herein and/or some aspects described can be excluded without departing from the methods and systems disclosed herein.

According to certain aspects, systems and non-transitory computer readable storage media are provided. The systems are configured with hardware configured to execute operations analogous to the methods of the present disclosure. The one or more non-transitory computer readable storage media comprise instructions that, when executed by one or more processors, cause the one or more processors to execute operations analogous to the methods of the present disclosure.

Similarly, where steps of a process are disclosed, those steps are described for purposes of illustrating the present methods and systems and are not intended to limit the disclosure to a particular sequence of steps. For example, the steps can be performed in differing order, two or more steps can be performed concurrently, additional steps can be performed, and disclosed steps can be excluded without departing from the present disclosure. Further, the disclosed processes can be repeated.

Although specific aspects were described herein, the scope of the technology is not limited to those specific aspects. One skilled in the art will recognize other aspects or improvements that are within the scope of the present technology. Therefore, the specific structure, acts, or media are disclosed only as illustrative aspects. The scope of the technology is defined by the following claims and any equivalents therein.

It is also to be appreciated that the embodiments presented herein are not mutually exclusive and that the various embodiments can be combined with another in any of a number of different manners.

1. A method, comprising:
capturing a plurality of evoked biological responses via electrodes configured to be inserted into a recipient; and
estimating a relative proximity of at least one of the electrodes to a target structure within the recipient based on the plurality of evoked biological responses.

2. The method of claim 1, wherein the plurality of evoked biological responses comprise a plurality of electrically evoked compound action potentials (ECAPs), and wherein estimating the relative proximity of at least one of the electrodes to the target structure within the recipient comprises:
determining a relative modiolar proximity of the at least one of the electrodes based on the plurality of ECAPs.

3. The method of claim 2, wherein estimating the modiolar proximity of at least one of the electrodes comprises:
estimating the modiolar proximity based on one or more ECAP threshold profiles determined from the plurality of ECAPs.

4. The method of claim 2, further comprising:
determining at least one Spread Of Excitation (SOE) function from the plurality of ECAPs, and
estimating the modiolar proximity of at least one of the electrodes based on the at least one SOE function.

5. The method of claim 4, wherein estimating the modiolar proximity of at least one of the electrodes based on the at least one SOE function, comprises:
analyzing a location of a peak of excitation associated with at least one SOE function relative to a location at which probe stimulation is delivered to obtain the at least one SOE function.

6. The method of claim 4, wherein estimating the modiolar proximity of at least one of the electrodes based on the at least one SOE function, comprises:
analyzing a level of excitation spread associated with at least one SOE function.

7. The method of claim 2, further comprising:
estimating the modiolar proximity of at least one of the electrodes based on the plurality of ECAPs and one or more electrical measurements.

8. The method of claim 7, wherein the one or more electrical measurements include impedance measurements.

9. The method of claim 7, wherein the one or more electrical measurements include voltage or current measurements.

10. The method of claim 2, further comprising:
estimating the modiolar proximity of at least one of the electrodes based on one or more ECAP amplitude growth functions determined from the plurality of ECAPs.

11. The method of claim 2, wherein estimating the modiolar proximity of at least one of the electrodes comprises:
analyzing the plurality of ECAPs relative to one another to estimate the modiolar proximity.

12. The method of claim 2, wherein estimating the modiolar proximity of at least one of the electrodes comprises:
analyzing the plurality of ECAPs relative to normative population data to estimate the modiolar proximity.

13. One or more non-transitory computer readable storage media comprising instructions that, when executed by one or more processors, cause the one or more processors to:
perform, with one or more of a plurality of electrodes configured to be inserted into a body chamber of a recipient, neural response telemetry measurements to obtain a plurality of evoked biological responses; and
use the plurality of evoked biological responses to estimate a relative proximity of the electrodes to a wall of the body chamber of the recipient.

14. The one or more non-transitory computer readable storage media of claim 13, wherein the instructions executable to use the plurality of evoked biological responses to estimate a relative proximity of the electrodes to a wall of the body chamber comprise instructions executable to:
determine a modiolar proximity of at least a first one of the plurality electrodes relative to a modiolar proximity of at least a second of the plurality of electrodes.

15. The one or more non-transitory computer readable storage media of claim 13, wherein the instructions executable to perform neural response telemetry measurements to obtain a plurality of evoked biological responses comprise instructions executable to:
perform neural response telemetry measurements to obtain a plurality of electrically evoked compound action potentials (ECAPs).

16. The one or more non-transitory computer readable storage media of claim 15, wherein the instructions executable to use the plurality of evoked biological responses to estimate a relative proximity of the electrodes to a wall of the body chamber comprise instructions executable to:
estimate the relative proximity of the electrodes to the wall of the body chamber based on one or more ECAP threshold profiles determined from the plurality of ECAPs.

17. The one or more non-transitory computer readable storage media of claim 15, further comprising instructions executable to:
determine at least one Spread Of Excitation (SOE) function from the plurality of ECAP, and
estimate the relative proximity of the electrodes to the wall of the body chamber based on the at least one SOE function.

18. The one or more non-transitory computer readable storage media of claim 17, wherein the instructions executable to estimate the relative proximity of the electrodes to the wall of the body chamber based on the at least one SOE function, comprise instructions executable to:
analyze a location of a peak of excitation associated with at least one SOE function relative to a location at which probe stimulation is delivered to obtain the at least one SOE function.

19. The one or more non-transitory computer readable storage media of claim 17, wherein the instructions executable to estimate the relative proximity of the electrodes to the wall of the body chamber based on the at least one SOE function, comprise instructions executable to:
analyze a level of excitation spread associated with at least one SOE measurement.

20. The one or more non-transitory computer readable storage media of claim 15, further comprising instructions executable to:
estimate the relative proximity of the electrodes to the wall of the body chamber based on one or more ECAP amplitude growth functions determined from the plurality of ECAPs.

21. The one or more non-transitory computer readable storage media of claim 13, further comprising instructions executable to:
estimate the relative proximity of the electrodes to the wall of the body chamber based on the plurality of evoked biological responses and one or more electrical measurements.

22. The one or more non-transitory computer readable storage media of claim 13, wherein the instructions executable to use the plurality of evoked biological responses to estimate a relative proximity of the electrodes to a wall of the body chamber comprise instructions executable to:
analyze the plurality of evoked biological responses relative to one another to estimate the relative proximity of the electrodes to a wall of the body chamber.

23. The one or more non-transitory computer readable storage media of claim 13, wherein the instructions executable to use the plurality of evoked biological responses to estimate a relative proximity of the electrodes to a wall of the body chamber comprise instructions executable to:
analyze the plurality of evoked biological responses relative to normative population data to estimate a relative proximity of the electrodes to a wall of the body chamber.

24. The one or more non-transitory computer readable storage media of claim 13, wherein the instructions executable to use the plurality of evoked biological responses to estimate a relative proximity of the electrodes to a wall of the body chamber comprise instructions executable to:
analyze the plurality of evoked biological responses to estimate a relative proximity of the electrodes to a modiolar wall of the body chamber.

25-31. (canceled)

32. A system, comprising:
one or more electrodes configured to record a plurality of evoked biological responses from a recipient; and
one or more processors configured to analyze the plurality of evoked biological responses relative to one another to determine a relative proximity of the electrodes to a target structure within the recipient.

33. The system of claim 32, wherein to analyze the evoked biological responses relative to one another to determine the relative proximity of at least one of the electrodes to the target structure within the recipient, the one or more processors are configured to:
analyze the evoked biological responses relative to one another to estimate the relative proximity of the at least one of the electrodes to a modiolus of a cochlea.

34. The system of claim 32, wherein the plurality of evoked biological responses comprise electrically evoked compound action potentials (ECAPs).

35. The system of claim 34, wherein the one or more processors are configured to:
determine at least one Spread Of Excitation (SOE) function from the plurality of ECAPs, and
analyze the SOE function to determine the relative proximity of at least one of the electrodes to a target structure within the recipient.

36. The system of claim 35, wherein to analyze the SOE function to determine the relative proximity of at least one of the electrodes to a target structure within the recipient, the one or more processors are configured to:
analyze a location of a peak of excitation associated with at least one SOE function relative to a location at which probe stimulation is delivered to obtain the at least one SOE function.

37. The system of claim 35, wherein to analyze the SOE function to determine the relative proximity of at least one of the electrodes to a target structure within the recipient, the one or more processors are configured to:
analyze a level of excitation spread associated with at least one SOE function.
